(19) United States
(12) Patent Application Publication
(10) Pub. No.: US 2026/0306418 A1
(43) Pub. Date: Oct. 8, 2026

(54) SILICA-BASED NANOPARTICLES FOR SUSTAINED RELEASE OF HYDROPHOBIC DRUG, PREPARATION METHOD THEREFOR, AND TRANSDERMAL DRUG DELIVERY SYSTEM COMPRISING SAME

(71) Applicant: MPIOS INC., Gunpo-si, Gyeonggi-do (KR)

(72) Inventors: Byoung Whan SOH, Suwon-si (KR); Sun Jeong PARK, Uiwang-si (KR)

(73) Assignee: MPIOS INC., Gunpo-si, Gyeonggi-do (KR)

(21) Appl. No.: 19/483,803

(22) PCT Filed: May 13, 2024
(86) PCT No.: PCT/KR2024/006490

§ 371 Date: Jan. 27, 2026

(30) Foreign Application Priority Data
May 15, 2023 (KR) 10-2023-0062719

(51) Int. Cl. A61K 9/51; A61K 9/00; A61K 31/192

(52) U.S. Cl. CPC A61K 9/5115; A61K 9/5192; A61K 31/192; A61K 9/0014

(57) ABSTRACT

The purpose of the present invention is to implement a hydrophobic drug as a transdermal drug delivery system (TDDS). The present invention relates to: silica-based nanoparticles for the sustained release of a hydrophobic drug, the silica-based nanoparticles comprising hollow nanoparticles that include a hollow core layer and a silica matrix shell layer, and the hydrophobic drug loaded into the hollow nanoparticles; a method for preparing same; and a transdermal drug delivery system comprising same.

### TECHNICAL FIELD

The present invention relates to silica-based nanoparticles for sustained release of a hydrophobic drug, a preparation method therefor, and a transdermal drug delivery system including the same.

### BACKGROUND ART

Drug administration methods include oral administration and parenteral administration such as intravenous and subcutaneous injection. Oral administration is a relatively easy way to administer a drug in the form of a tablet, a capsule, a liquid, and the like, and is an inexpensive and convenient administration method because it does not require administration equipment or the assistance of specialized personnel, and the like. However, oral administration has a disadvantage in that drugs taken orally are affected by gastric acidity, gastrointestinal motility, and the like and the first-pass effect can cause the drug concentration in the bloodstream to be inconsistent, which can lead to a decrease in therapeutic efficacy, and an additional disadvantage is the possibility of gastrointestinal diseases such as gastric ulcers and gastric perforation when nonsteroidal anti-inflammatory drugs, narcotic analgesics, and antibiotics are orally administered.

Parenteral administration has disadvantages such as allergic reactions such as hives and edema, tissue damage, pain, bleeding, discomfort during administration, and the risk of embolism when air bubbles are present in the syringe, and has a disadvantage in that the risk of infection at the injection site or in the bloodstream may cause serious complications such as sepsis.

Meanwhile, a transdermal drug delivery system (TDDS) has remarkably developed since the first motion sickness treatment patch was approved by the US FDA in 1979, thereby overcoming the disadvantages of oral and parenteral administration. The TDDS directly delivers a drug through the skin and thus does not have the risk of generating the first-pass effect and gastrointestinal disorders, and as a non-invasive method, it offers the advantage of eliminating the discomfort and pain during administration and the risk of embolism or sepsis.

However, paradoxically, commercial patches and ointments do not exhibit excellent performance compared to oral or parenteral administration methods in terms of sustained release and sustainability, which can be the greatest feature of TDDS. Currently, most commercial transdermal drug delivery carriers are developed in the form of hydrogels, microparticles, microspheres, and the like based on polymeric substances such as PEG, PVA, HA, and chitosan, but have disadvantages in that the particle size distribution is non-uniform due to the nature of polymers, there is a risk of side effects due to excessive drug release at the initial stage of administration caused by swelling or polymer chain breakage, and their efficacy lasts for a short period of time.

### RELATED ART DOCUMENTS

### Patent Documents

(Patent Document 1) Korean Patent Publication No. 10-2020-0091682 (Jul. 31, 2020)

### DISCLOSURE

### Technical Problem

The purpose of the present invention is to implement a hydrophobic drug as a transdermal drug delivery system (TDDS). The present invention has been made in an effort to provide silica-based nanoparticles for the sustained release of a hydrophobic drug, the silica-based nanoparticles including hollow nanoparticles that include a hollow core layer and a silica matrix shell layer, and the hydrophobic drug loaded into the hollow nanoparticles; and the like.

However, the technical problem to be achieved by the present invention is not limited to the aforementioned problems, and other problems that are not mentioned may be clearly understood by a person skilled in the art from the following description.

### Technical Solution

The present invention provides silica-based nanoparticles for the sustained release of a hydrophobic drug, the silica-based nanoparticles including hollow nanoparticles that include a hollow core layer and a silica matrix shell layer, and a hydrophobic drug loaded into the hollow nanoparticles.

The hydrophobic drugs may be BCS Class II or BCS Class IV.

The hollow core layer may have a size of 100 to 600 nm, and the hollow nanoparticles may have a size of 200 to 900 nm.

In one embodiment of the present invention, provided is a method for preparing silica-based nanoparticles for the sustained release of a hydrophobic drug, the method including: (a) stirring a solvent and a surfactant to prepare a homogeneous phase; (b) adding a silica precursor to the homogeneous phase prepared in step (a) to carry out a primary reaction, and then preparing nanoparticles through a hydrothermal synthesis reaction at a temperature higher than the primary reaction temperature; (c) drying the nanoparticles prepared in step (b) and then calcining the dried nanoparticles to prepare hollow nanoparticles; and (d) impregnating the hollow nanoparticles prepared in step (c) with a hydrophobic drug solution to load the hydrophobic drug solution into the hollow nanoparticles.

In step (a), the solvent may be one or more selected from the group consisting of water, a C1 to C4 lower alcohol, and ammonia water, and the surfactant may be one or more selected from the group consisting of cetyltrimethylammonium bromide (CTAB), 1,3,5-trimethylbenzene (1,3,5-TMB), Pluronic P123 (P123), Pluronic F127 (F127), benzene, and toluene.

In step (a), the content of the surfactant may be 0.5 g to 50 g based on 1 L of the solvent.

In step (b), the silica precursor may be tetraethyl orthosilicate (TEOS) or tetramethyl orthosilicate (TMOS).

In step (b), the hydrothermal synthesis reaction may be carried out at a temperature of 80 to 200° C. for 24 to 168 hours.

In step (c), the drying may be performed at a temperature of 80 to 120° C. for 1 to 24 hours, and the calcination may be performed at a temperature of 300 to 700° C. for 3 to 12 hours.

In another embodiment of the present invention, provided is a transdermal drug delivery system including the silica-based nanoparticles for the sustained release of a hydrophobic drug.

### Advantageous Effects

The silica-based nanoparticles for the sustained release of a hydrophobic drug according to the present invention are characterized by including hollow nanoparticles including a hollow core layer and a metal-infiltrated silica matrix shell layer, and a hydrophobic drug loaded into the hollow nanoparticles.

In the present invention, since hollow nanoparticles are prepared from silica, an inorganic material, and used as a drug delivery platform, they are easily hydrated or do not react with water under hydrophilic conditions, and their physical properties, such as specific surface area and pore volume can be flexibly changed, thereby enabling a large amount of the drug to be loaded.

In particular, since it is possible to improve release sustainability while preventing the initial excessive release of a hydrophobic drug, there is an advantage in that the hydrophobic drug can be implemented as a transdermal drug delivery system (TDDS).

### DESCRIPTION OF DRAWINGS

FIG. 1A to 1C are TEM photographs of the silica nanoparticles of Examples 1 to 3.

FIG. 2 is a set of schematic views showing an aspect in which a drug is released from the mesoporous silica particles of the present invention.

FIG. 3A is a graph showing the results of analyzing the drug release amount of ibuprofen as a hydrophobic drug loaded into the silica nanoparticles in Examples 1 to 3, FIG. 3B is a graph showing the results of analyzing the drug release amount of ibuprofen salt as a hydrophilic drug loaded into the silica nanoparticles in Example 1, and FIG. 3C is a graph showing the results of analyzing the drug release amount of loxoprofen salt as a hydrophilic drug loaded into the silica nanoparticles in Example 2.

FIG. 4 is a graph showing the results of analyzing the drug release amount of ketoprofen as a hydrophobic drug loaded into the silica nanoparticles in Example 3.

### BEST MODE

The present inventors confirmed that hollow nanoparticles were prepared by adding a silica precursor to a homogeneous phase in which an optimal solvent and an optimal surfactant were stirred, and then performing hydrothermal synthesis, and then various drugs were successfully loaded, and a hydrophobic drug could be sustainably released, thereby completing the silica-based nanoparticles for the sustained release of a hydrophobic drug according to the present invention.

Hereinafter, the present invention will be described in detail.

### Silica-Based Nanoparticles for Sustained Release of Hydrophobic Drug and Transdermal Drug Delivery System

The present invention provides silica-based nanoparticles for the sustained release of a hydrophobic drug, the silica-based nanoparticles including hollow nanoparticles that include a hollow core layer and a silica matrix shell layer, and a hydrophobic drug loaded into the hollow nanoparticles.

Further, the present invention provides a transdermal drug delivery system including the silica-based nanoparticles for the sustained release of a hydrophobic drug.

First, the silica-based nanoparticles for the sustained release of a hydrophobic drug according to the present invention include hollow nanoparticles including a hollow core layer and a silica matrix shell layer.

Specifically, the hollow nanoparticles include a hollow core layer and a silica matrix shell layer.

The hollow core layer may be a layer formed by removing the surfactant located in the core during the drying and calcination of the nanoparticles.

The hollow core layer may have a size of 100 to 600 nm, preferably 120 to 500 nm, or 150 to 300 nm, but is not limited thereto. The size of the hollow core layer may be controlled by the type and content of the surfactant. In order to effectively prevent the initial excessive release of a hydrophobic drug to be described below, the size of the hollow core layer is most preferably controlled to 150 to 200 nm.

The silica matrix shell layer is a layer formed by hydrothermal synthesis of a silica precursor, and a plurality of pores may be formed.

The hollow nanoparticles may have a size of 200 to 900 nm, preferably 200 to 800 nm, 200 to 700 nm, 200 to 600 nm, 200 to 500 nm, or 250 to 400 nm, but are not limited thereto.

Meanwhile, the hollow nanoparticles may have a specific surface area of 100 to 1,000 m²/g, preferably 100 to 300 m²/g, but are not limited thereto. In addition, the hollow nanoparticles may have a pore volume and a pore size of 0.1 to 2.0 cm³/g and 1 to 15 nm, respectively, and preferably, a pore volume and a pore size of 0.2 to 1.0 cm³/g and 1 to 10 nm, respectively, but are not limited thereto.

Next, the silica-based nanoparticles for the sustained release of a hydrophobic drug according to the present invention include a hydrophobic drug, which is characterized by being loaded into the hollow nanoparticles.

The hydrophobic drug is in a form having low solubility, and is preferably BCS Class II or BCS Class IV, but is not limited thereto. The BCS Class described above is a methodology for classifying drugs according to their solubility and permeability, and in this case, low solubility means that the volume of an aqueous solution sufficient to dissolve the maximum single dose of a general oral solid preparation containing the same main ingredient in a pH range of 1.2 to 6.8 exceeds 250 mL. BCS Class II refers to a group that has low solubility but high permeability, and BCS Class IV refers to a group that has both low solubility and low permeability. Specifically, the hydrophobic drug may be BCS Class II such as ibuprofen, ketoprofen, loxoprofen, diclofenac, rotigotine, griseofulvin, itraconazole, ketoconazole, tacrolimus, nifedipine, and carbamazepine, or BCS Class IV such as furosemide, hydrochlorothiazide, cyclosporin A, paclitaxel, albendazole, azathioprine, and didanosine. The salt forms of these hydrophobic drugs may be classified as hydrophilic drugs.

The hydrophobic drug may be efficiently loaded into the hollow nanoparticles and then have improved release sustainability without initial excessive release through interaction with the hollow nanoparticles having enhanced hydrophobicity.

Meanwhile, the silica-based nanoparticles for the sustained release of a hydrophobic drug according to the present invention have the advantage of improving release sustainability while preventing the initial excessive release of the hydrophobic drug in implementing the hydrophobic drug as a transdermal drug delivery system.

Specifically, the amount of hydrophobic drug loaded may be 10 to 90 wt %, preferably 30 to 80 wt % or 40 to 60 wt %, compared to the silica nanoparticles, but is not limited thereto.

When the silica nanoparticles are subjected to a dissolution test under conditions of a temperature of 32° C. and a paddle speed of 600 rpm in an SBF solution in accordance with the in vitro skin absorption test guidelines (Ministry of Food and Drug Safety), the dissolution rate of the hydrophobic drug (particularly ibuprofen and ketoprofen) after 40 hours may be 60% or less, preferably 10 to 50% or 20 to 40%, but is not limited thereto.

As discussed above, the silica-based nanoparticles for the sustained release of a hydrophobic drug according to the present invention are characterized by including hollow nanoparticles including a hollow core layer and a metal-infiltrated silica matrix shell layer, and a hydrophobic drug loaded into the hollow nanoparticles.

In the present invention, since hollow nanoparticles are prepared from silica, an inorganic material, and used as a drug delivery platform, they are easily hydrated or do not react with water under hydrophilic conditions, and their physical properties, such as specific surface area and pore volume can be flexibly changed, thereby enabling a large amount of the drug to be loaded.

In particular, since it is possible to improve release sustainability while preventing the initial excessive release of a hydrophobic drug, there is an advantage in that the hydrophobic drug can be implemented as a transdermal drug delivery system (TDDS).

### Method for Preparing Silica-Based Nanoparticles for Sustained Release of Hydrophobic Drug

The present invention provides a method for preparing silica-based nanoparticles for the sustained release of a hydrophobic drug, the method including: (a) stirring a solvent and a surfactant to prepare a homogeneous phase; (b) adding a silica precursor to the homogeneous phase prepared in step (a) to carry out a primary reaction, and then preparing nanoparticles through a hydrothermal synthesis reaction at a temperature higher than the primary reaction temperature; (c) drying the nanoparticles prepared in step (b) and then calcining the dried nanoparticles to prepare hollow nanoparticles; and (d) impregnating the hollow nanoparticles prepared in step (c) with a hydrophobic drug solution to load the hydrophobic drug solution into the hollow nanoparticles.

First, the method for preparing silica-based nanoparticles for the sustained release of a hydrophobic drug according to the present invention includes stirring a solvent and a surfactant to prepare a homogeneous phase [step (a)].

The solvent is used to uniformly dissolve the surfactant, may be one or more selected from the group consisting of water, a C1 to C4 lower alcohol, and ammonia water, and preferably includes all of water, a C1 to C4 lower alcohol (for example, ethanol), and ammonia water, but is not limited thereto. Specifically, the volume ratio of the water, C1 to C4 lower alcohol (for example, ethanol), and ammonia water may be 10:5:1 to 50:15:1, preferably 20:8:1 to 30:10:1, but is not limited thereto.

Furthermore, the surfactant may be one or more selected from the group consisting of cetyltrimethylammonium bromide (CTAB), 1,3,5-trimethylbenzene (1,3,5-TMB), Pluronic P123 (P123), Pluronic F127 (F127), benzene, and toluene, and is preferably two or more selected from the group including cetyltrimethylammonium bromide (CTAB), 1,3,5-trimethylbenzene (1,3,5-TMB), and Pluronic P123 (P123), but is not limited thereto.

Specifically, when the surfactant includes three types, such as 1,3,5-TMB, CTAB, and P123, the weight ratio of 1,3,5-TMB, CTAB, and P123 may be 1:10:30, preferably 1:2:20 to 1:3:30, but is not limited thereto. In this case, the total content of these surfactants may be 0.5 g to 10 g based on 1 L of the solvent. Meanwhile, when the surfactant includes two types, such as 1,3,5-TMB and P123, the ratio may be 1:10 to 1:20. In this case, the total content of these surfactants may be 10 g to 50 g based on 1 L of the solvent.

The type and content of such surfactants may control the size of the hollow core layer and the size of the nanoparticles.

Next, the method for preparing silica-based nanoparticles for the sustained release of a hydrophobic drug according to the present invention includes adding a silica precursor to the homogeneous phase prepared above to carry out a primary reaction, and then preparing nanoparticles through a hydrothermal synthesis reaction at a temperature higher than the primary reaction temperature [step (b)].

The silica precursor may be tetraethyl orthosilicate (TEOS) or tetramethyl orthosilicate (TMOS).

The primary reaction may be carried out at room temperature for 1 to 10 hours, and may be carried out at room temperature for 2 to 7 hours.

After the primary reaction, the hydrothermal synthesis reaction may be carried out in an autoclave, may be carried out at a temperature of 80 to 200° C. for 24 to 168 hours, and is preferably carried out at a temperature of 80 to 180° C., 80 to 160° C., 80 to 140° C., 80 to 130° C., 100 to 200° C., 100 to 180° C., 100 to 160° C., 100 to 140° C., 100 to 130° C., 120 to 200° C., 120 to 180° C., 120 to 160° C., 120 to 140° C., or 120 to 130° C. for 36 hours, but is not limited thereto.

After the hydrothermal synthesis reaction, processes such as vacuum filtration and washing with distilled water may be added.

Next, the method for preparing silica-based nanoparticles for the sustained release of a hydrophobic drug according to the present invention includes drying the nanoparticles prepared above and then calcining the dried nanoparticles to prepare hollow nanoparticles [step (c)].

Through the drying and calcination, the surfactant located in the core may be removed, thereby forming the hollow core layer.

The drying may be performed at a temperature of 80 to 120° C. for 1 to 24 hours, and is preferably performed at a temperature of 90 to 110° C. for 6 to 24 hours, but is not limited thereto.

Thereafter, the calcination may be performed at 300 to 700° C. for 1 to 24 hours, and is preferably performed at 400 to 600° C. for 1 to 12 hours, but is not limited thereto.

Next, the method for preparing silica-based nanoparticles for the sustained release of a hydrophobic drug according to the present invention includes impregnating the hollow nanoparticles prepared above with a hydrophobic drug solution to load the hydrophobic drug solution into the hollow nanoparticles [step (d)].

The hydrophobic drug solution may include a solvent (for example, ethanol) capable of dissolving the hydrophobic drug, and the impregnation may be performed using an incipient wetness impregnation method or excess water impregnation method known in the art.

Hereinafter, the present invention will be described in detail through examples and experimental examples. However, the following examples and experimental examples are for illustrative purposes only, and the scope of the present invention is not limited thereto.

### Example 1. Preparation of Silica Nanoparticles (1)

125 ml of distilled water, 75 ml of ethanol, and 15 ml (25 to 28%) of ammonia water as solvents, and 0.105 g of cetyltrimethylammonium bromide (CTAB), 1.045 g of Pluronic P123 (P123), and 0.042 g of 1,3,5-trimethylbenzene (1,3,5-TMB) as surfactants were put into a round flask and stirred for 3 hours to prepare a solution having a homogeneous phase, and 10 ml of a silica precursor tetraethoxysilane (TEOS) was added and allowed to react at room temperature for 5 hours, and then the solution was treated in an autoclave under conditions of 130° C. and 48 hours. The treated solution was filtered under reduced pressure and washed with distilled water to obtain silica nanoparticles. The obtained silica nanoparticles were dried in an oven at 100° C. for 12 hours and then calcined at 500° C. for 6 hours.

### Example 2. Preparation of Silica Nanoparticles (2)

125 ml of distilled water, 75 ml of ethanol, and 15 ml (25 to 28%) of ammonia water as solvents, and 0.3 g of CTAB, 0.85 g of P123, and 0.051 g of 1,3,5-TMB as surfactants were put into a round flask and stirred for 3 hours to prepare a solution having a homogeneous phase, and 10 ml of a silica precursor TEOS was added and allowed to react at room temperature for 5 hours, and then the solution was treated in an autoclave under conditions of 130° C. and 48 hours. The treated solution was filtered under reduced pressure and washed with distilled water to obtain silica nanoparticles. The obtained silica nanoparticles were dried in an oven at 100° C. for 12 hours and then calcined at 500° C. for 6 hours.

### Example 3. Preparation of Silica Nanoparticles (3)

125 ml of distilled water, 75 ml of ethanol, and 15 ml (25 to 28%) of ammonia water as solvents, and 4.0 g of P123 and 0.3 g of 1,3,5-TMB as surfactants were put into a round flask and stirred for 3 hours to prepare a solution having a homogeneous phase, and 10 ml of a silica precursor TEOS was added and allowed to react at room temperature for 5 hours, and then the solution was treated in an autoclave under conditions of 130° C. and 48 hours. The treated solution was filtered under reduced pressure and washed with distilled water to obtain silica nanoparticles. The obtained silica nanoparticles were dried in an oven at 100° C. for 12 hours and then calcined at 500° C. for 6 hours.

### Experimental Example 1. Analysis of Silica Nanoparticles (1) to (3)

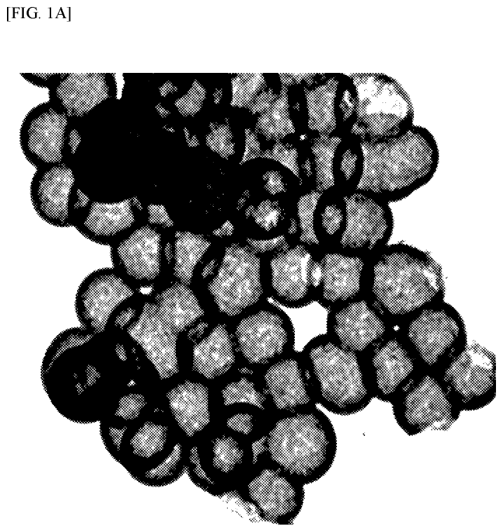

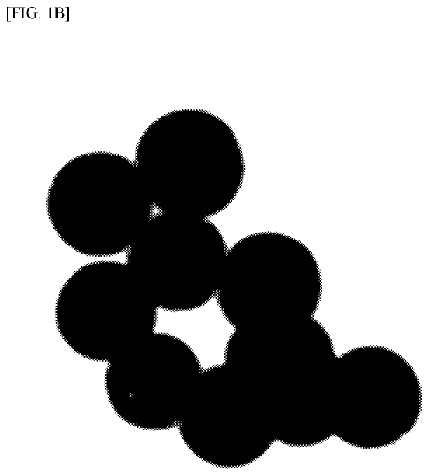

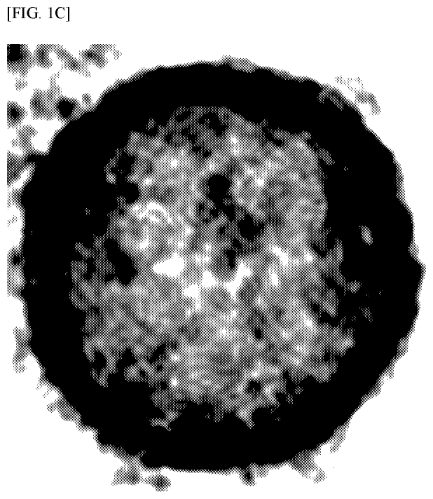

The particle size, hollow size, shell size, surface area, pore volume, and pore size of the silica nanoparticles of Examples 1 to 3 were measured and are shown in the following Table 1. Further, TEM photographs were taken, and are shown in FIGS. 1A to 1C.

TABLE 1

| | Particle | Hollow | Shell | Surface | Pore | Pore |
|---|---|---|---|---|---|---|
| Classi- | size | size | size | area | volume | size |
| fication | (nm) | (nm) | (nm) | (m²/g) | (cm³/g) | (nm) |
| Example 1 | 374 ± 25 | 182 ± 20 | 96 ± 15 | 282 | 0.27 | 7 |
| Example 2 | 383 ± 40 | 254 ± 35 | 64 ± 8 | 208 | 0.35 | 10 |
| Example 3 | 285 ± 75 | 197 ± 60 | 55 ± 18 | 190 | 0.2 | 6 |

### Experimental Example 2. Hydrophobic Drug (1) Loading Experiment

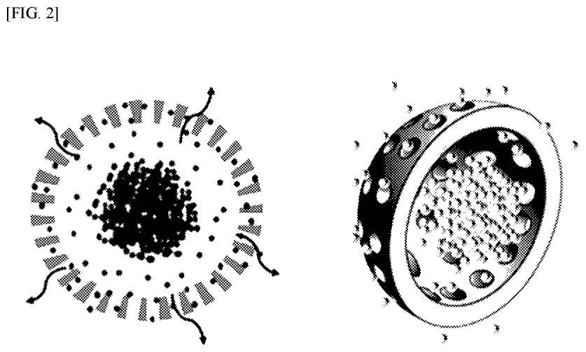

After ethanol was added to 1 g of ibuprofen as a hydrophobic drug to prepare a 20 ml solution, 1 g of the silica nanoparticles prepared in Example 1 or 2 was added and the resulting mixture was stirred at room temperature for 24 hours. Thereafter, the drug-loaded silica nanoparticles were filtered under reduced pressure and then washed with ethanol to obtain washed drug-loaded silica nanoparticles, and the washed drug-loaded silica nanoparticles were dried under reduced pressure at 60° C. for 4 hours. In this case, the drug loading amount was confirmed by measuring the concentration of the drug dissolved in a washing solution using a UV measurement device. A schematic view of the drug release pattern is shown in FIG. 2. The drug loading amount was measured and is shown in the following Table 2.

TABLE 2

| | Amount (g) of ibuprofen loaded per g of |
|---|---|
| Classification | silica |
| Example 1 | 0.408 |
| Example 2 | 0.571 |

### Experimental Example 3. Hydrophobic Drug (2) Loading Experiment

After ethanol was added to 1 g of ketoprofen as a hydrophobic drug to prepare a 20 ml solution, 1 g of the silica nanoparticles prepared in Example 3 was added and the resulting mixture was stirred at room temperature for 24 hours. Thereafter, the drug-loaded silica nanoparticles were filtered under reduced pressure and then washed with ethanol to obtain washed drug-loaded silica nanoparticles, and the washed drug-loaded silica nanoparticles were dried under reduced pressure at 60° C. for 4 hours. In this case, the drug loading amount was confirmed by measuring the concentration of the drug dissolved in a washing solution using a UV measurement device. The drug loading amount was measured and is shown in the following Table 2.

TABLE 3

| | Amount (g) of ketoprofen loaded per g of |
|---|---|
| Classification | silica |
| Example 3 | 0.554 |

### Experimental Example 4. Hydrophobic Drug (1) Release Experiment

After an appropriate amount of distilled water was added dropwise to about 0.004 g of silica nanoparticles of Examples 1 to 3 loaded with ibuprofen as a hydrophobic drug to prepare a mixture in the form of a paste, the paste was applied evenly to the top surface of a membrane (artificial skin, area: 1.13 cm²). The membrane to which silica paste was applied was placed on a Franz diffusion cell (FDC) and fixed using a clamp in order to prevent the membrane from moving. After a receptor chamber was filled with a simulated body fluid (SBF) solution, UV absorbance was measured by taking 3 ml of the solution at predetermined intervals while stirring the chamber under the conditions of 32° C. and 600 rpm to confirm the concentration of the released drug. In this case, the receptor chamber was filled with fresh SBF equal to the volume of the solution taken for sampling.

Meanwhile, in Comparative Example 1, an experiment was carried out in the same manner as in Example 1 using the silica nanoparticles of Example 1 loaded with ibuprofen salt, a hydrophilic drug. In addition, in Comparative Example 2, an experiment was carried out in the same manner as in Example 2 using the silica nanoparticles of Example 2 loaded with loxoprofen salt, a hydrophilic drug.

As a control, commercial products A and B (ibuprofen and loxoprofen) having the same area as that of the membrane (artificial skin, area: 1.13 cm²) were attached onto the membrane. An experiment was carried out in the same manner, except that the membrane with commercial products A and B attached thereto was placed on the FDC and fixed using a clamp to prevent the membrane from moving.

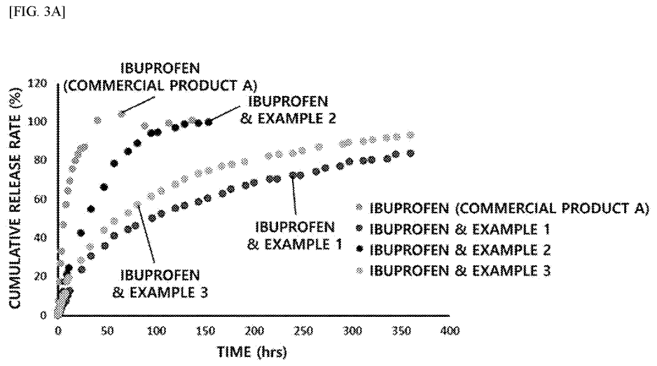

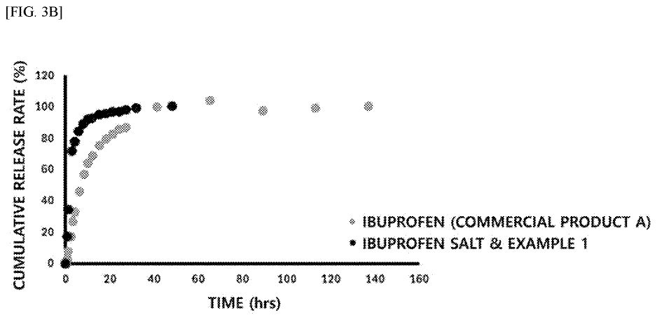

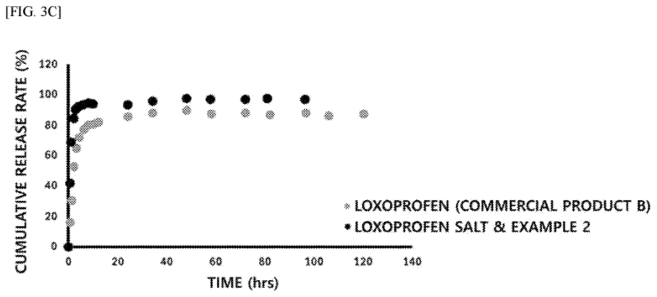

The drug release amount was measured and is shown in FIGS. 3A to 3C. As shown in FIGS. 3A to 3C, it was confirmed that in the in-vitro release experimental results for commercial products A and B, almost all of the drug was released within about 40 hours, but it was confirmed that ibuprofen, a hydrophobic drug loaded into the hollow mesoporous silica of Examples 1 and 2 of the present invention, showed initial sustained release without an initial burst and had release sustainability even after several hundred hours. That is, it was confirmed that ibuprofen as a hydrophobic drug loaded into the hollow mesoporous silica of Examples 1 and 2 of the present invention exhibits both sustained release and sustainability.

However, it was confirmed that in the case of Comparative Examples 1 and 2, there was a problem of an initial burst compared to commercial product A.

### Experimental Example 5. Hydrophobic Drug (2) Release Experiment

After an appropriate amount of distilled water was added dropwise to about 0.002 g of silica nanoparticles of Example 3 loaded with ibuprofen as a hydrophobic drug to prepare a mixture in the form of a paste, the paste was applied evenly to the top surface of a membrane (artificial skin, area: 1.13 cm²). The membrane to which silica paste was applied was placed on a Franz diffusion cell (FDC) and fixed using a clamp in order to prevent the membrane from moving. After a receptor chamber was filled with a simulated body fluid (SBF) solution, UV absorbance was measured by taking 3 ml of the solution at predetermined intervals while stirring the chamber under the conditions of 32° C. and 600 rpm to confirm the concentration of the released drug. In this case, the receptor chamber was filled with fresh SBF equal to the volume of the solution taken for sampling.

As a control, commercial product C (ketoprofen) having the same area as that of the membrane (artificial skin, area: 1.13 cm²) was attached onto the membrane. An experiment was carried out in the same manner, except that the membrane with commercial product C attached thereto was placed on the FDC and fixed using a clamp to prevent the membrane from moving.

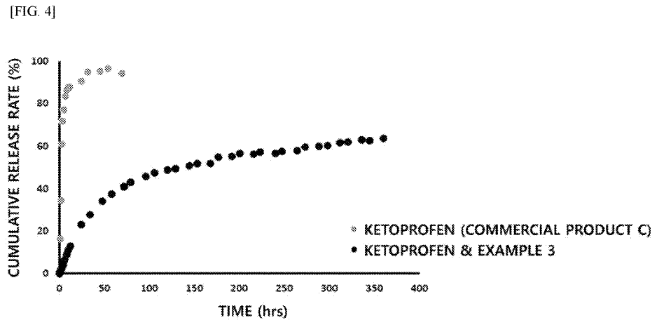

The drug release amount was measured and is shown in FIG. 4. As shown in FIG. 4, it was confirmed that in the in-vitro release experimental results for commercial product C, almost all of the drug was released within about 40 hours, but it was confirmed that ketoprofen, a hydrophobic drug loaded into the hollow mesoporous silica of Example 3 of the present invention, showed initial sustained release without an initial burst and had release sustainability even after several hundred hours. That is, it was confirmed that ketoprofen as a hydrophobic drug loaded into the hollow mesoporous silica of Example 3 of the present invention exhibits both sustained release and sustainability.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are exemplary in all aspects and not restrictive. For example, each constituent element described as a singular form may be implemented in a distributed form, and similarly, constituent elements described as being distributed may be implemented in a combined form.

The scope of the present invention is indicated by the following claims, and all changes or modified forms derived from the meaning and scope of the claims and their equivalent concepts should be interpreted as falling within the scope of the present invention.

1. Silica-based nanoparticles for the sustained release of a hydrophobic drug, the silica-based comprising hollow nanoparticles that comprise a hollow core layer and a silica matrix shell layer, and
a hydrophobic drug loaded into the hollow nanoparticles.

2. The silica-based nanoparticles of claim 1, wherein the hydrophobic drug is BCS Class II or BCS Class IV.

3. The silica-based nanoparticles of claim 1, wherein the hollow core layer has a size of 100 to 600 nm, and the hollow nanoparticles have a size of 200 to 900 nm.

4. A method for preparing silica-based nanoparticles for the sustained release of a hydrophobic drug, the method including:
(a) stirring a solvent and a surfactant to prepare a homogeneous phase;
(b) adding a silica precursor to the homogeneous phase prepared in step (a) to carry out a primary reaction, and then preparing nanoparticles through a hydrothermal synthesis reaction at a temperature higher than the primary reaction temperature;
(c) drying the nanoparticles prepared in step (b) and then calcining the dried nanoparticles to prepare hollow nanoparticles; and
(d) impregnating the hollow nanoparticles prepared in step (c) with a hydrophobic drug solution to load the hydrophobic drug solution into the hollow nanoparticles.

5. The method of claim 4, wherein in step (a), the solvent is one or more selected from the group consisting of water, a C1 to C4 lower alcohol, and ammonia water, and the surfactant may be one or more selected from the group consisting of cetyltrimethylammonium bromide (CTAB), 1,3,5-trimethylbenzene (1,3,5-TMB), Pluronic P123 (P123), Pluronic F127 (F127), benzene, and toluene.

6. The method of claim 4, wherein in step (a), a content of the surfactant is 0.5 g to 50 g based on 1 L of the solvent.

7. The method of claim 4, wherein in step (b), the silica precursor is tetraethyl orthosilicate (TEOS) or tetramethyl orthosilicate (TMOS).

8. The method of claim 4, wherein in step (b), the hydrothermal synthesis reaction is carried out at a temperature of 80 to 200° C. for 24 to 168 hours.

9. The method of claim 4, wherein in step (c), the drying is performed at a temperature of 80 to 120° C. for 1 to 24 hours, and the calcination is performed at a temperature of 300 to 700° C. for 3 to 12 hours.

10. A transdermal drug delivery system comprising the silica-based nanoparticles of claim 1.
